(19) United States
(12) Patent Application Publication
(10) Pub. No.: US 2022/0008325 A1
(43) Pub. Date: Jan. 13, 2022

(54) WHITE PINE BARK EXTRACT FOR DECREASING ENDOTHELIN-1 SECRETION, STEM CELL FACTOR SYNTHESIS AND PROTEIN CARBONYLATION

(71) Applicant: International Flavors & Fragrances Inc., New York, NY (US)

(72) Inventors: Mariana ROYER, Union Beach, NJ (US); Joan ATTIA-VIGNEAU, Union Beach, NJ (US); Estelle LOING, Union Beach, NJ (US)

(21) Appl. No.: 17/295,934

(22) PCT Filed: Nov. 26, 2019
(86) PCT No.: PCT/US2019/063183

§ 371 Date: May 21, 2021

Related U.S. Application Data
(60) Provisional application No. 62/774,669, filed on Dec. 3, 2018.

(51) Int. Cl. A61K 8/9767; A61K 8/34; A61K 8/49; A61Q 19/00; A61Q 19/02

(52) U.S. Cl. CPC A61K 8/9767; A61K 8/347; A61Q 19/02; A61Q 19/00; A61K 8/498

(57) ABSTRACT

A method for decreasing endothelin-1 secretion, stem cell factor synthesis and/or protein carbonylation in a keratinocyte using an aqueous extract of Pinus strobus bark is provided.

### BACKGROUND

Melanogenesis by definition is the production of the melanin pigments by cells called melanocytes. Melanocytes in the skin are surrounded by keratinocytes (one melanocyte is surrounded by approximately 36 keratinocytes), to which they transfer their melanin pigment. Melanocytes and keratinocytes interact with each other extensively following extrinsic stimuli (e.g., ultraviolet radiation (UVR) and drugs) or intrinsic stimuli (e.g., keratinocytes and fibroblasts, endocrine, inflammatory and neuronal cells). In response to UVR, keratinocytes produce several factors, with paracrine action on melanocytes. More specifically, keratinocyte-derived cytokines including basic fibroblast growth factor, endothelin-1, α-melanocyte-stimulating hormone (α-MSH), stem cell factor (SCF), and nitric oxide have been shown to be up-regulated in their production and secretion/release following UVB irradiation and can act as mitogens and/or melanogens to stimulate proliferation and melanogenesis of human melanocytes.

UV protection and skin whitening activities associated with a Pinus strobus extract have been suggested (KR 100860604 and US 2010/0129304). However, these documents do not suggest any effect on keratinocytes.

### SUMMARY OF THE INVENTION

This invention provides a method for decreasing endothelin-1 secretion, stem cell factor synthesis and/or protein carbonylation in a keratinocyte by contacting the keratinocyte with an effective amount of an aqueous extract of Pinus strobus bark. In some embodiments, endothelin-1 secretion is decreased by at least 30%; stem cell factor synthesis is decreased by at least 50%; protein carbonylation is decreased by at least 10%. In certain embodiments, the aqueous extract of Pinus strobus bark includes 4% flavonoids, 15% polyphenols, 0.1% trans-resveratrol, and 0.1% catechin. In other embodiments, the aqueous extract of P. strobus bark is in the form of a cream, ointment, foam, lotion, plaster, gel, solution or emulsion.

### BRIEF DESCRIPTION OF THE DRAWINGS

FIG. 1 shows that Pinus strobus bark extract modulates melanin synthesis in murine melanocytes after α-MSH stimulation.

FIG. 2 shows that Pinus strobus bark extract inhibits endothelin-1 synthesis in human keratinocytes. Keratinocyte cell culture without treatment (pinus bark extract) served as the control. ***p<0.001.

FIG. 3 shows that Pinus strobus bark extract inhibits stem cell factor (SCF) synthesis in human keratinocytes. Keratinocyte cell culture without treatment (pinus bark extract) served as the control. ***p<0.001.

FIG. 4 shows that Pinus strobus bark extract inhibits protein carbonylation in human keratinocytes. HNE (4-Hydroxynonenal) was used as a reference activator of protein carbonylation at 20 μM. DNPH (2,4-Dinitrophenyl hydrazine) was used as detector of carbonylation (1 μL for 0.5 μg/μL of protein). *, p<0.05; **, p<0.01.

FIG. 5 is a schematic of the ex vivo protocol design to demonstrate melanin synthesis inhibition by the Pinus strobus bark extract using a reconstructed human epidermis (RHE).

FIG. 6 shows that Pinus strobus bark extract inhibits skin pigmentation in an ex vivo model of human skin. Ex vivo culture treated by the placebo formula (formula without the Pinus Bark Extract) was used as a control. **p<0.01, ***p<0.001.

### DETAILED DESCRIPTION OF THE INVENTION

It has now been found that an aqueous extract of Pinus strobus bark modulates keratinocyte-derived cytokines involved in the proliferation and melanogenesis of human melanocytes. In particular, the Pinus strobus bark extract decreases endothelin-1 secretion, stem cell factor synthesis and/or protein carbonylation in keratinocytes. Accordingly, the present invention provides a method for decreasing endothelin-1 secretion, stem cell factor synthesis or protein carbonylation in a keratinocyte by contacting the keratinocyte with an effective amount of an aqueous extract of Pinus strobus bark. In light of these activities, the Pinus strobus bark extract is of use in maintaining skin homeostasis and modulating complexion by improving whitening, opacity and redness to provide more radiant skin.

As used herein, the terms “aqueous extract of Pinus strobus bark” and “Pinus strobus bark extract” are used interchangeably to refer to material that has been extracted from the bark of Pinus strobus with water. The Pinus strobus bark extract of this invention may be obtained by grinding the dried bark of Pinus strobus (commonly referred to as white pine) to a particle size of less than 0.5 mm and subjecting the ground material to hot water extraction. The ground bark may be extracted with water at a w/w ratio of 1 part plant material to 5-20 parts water, or more preferably at a 1:16 ratio. Initially, the water that is added to the ground bark is at a temperature above 70° C., 75° C. or 80° C. and is preferably at 85° C. Indeed, temperatures below 70° C. and above 90° C. have been shown to negatively impact the extraction of quercetins and kaempferols from mulberry (Tchabo, eta 1. (2018) Intern. J. Food Proper. 21(1):717-732). Furthermore, water extraction of proanthocyanidins from Pinus radiata bark has been shown to be significantly dependent on temperature, with maximum values achieved at 80° C. (Ku, et al. (2011) Forest Prod. J. 61(4):321-5).

Ideally, extraction of the Pinus strobus is allowed to proceed about 0.5 to 5 hours, preferably about 1 hour, with agitation and no supplemental heat. Once particulate bark material is removed (e.g., by filtration and/or centrifugation), the aqueous extract is dried. As the drying method, spray drying, freeze-drying, and the like may be mentioned without limitation thereto. While the extract may be used in its dry form and incorporated directly into a cosmetic formulation, ideally, the extract is dissolved in a mixture of water and glycerin, e.g., a 60-90% glycerin solution, or more preferably a 70-80% glycerin solution. In particular, 2.5% extract (solid content) is dissolved in 20% water and 77.5% glycerin.

The activity of a Pinus strobus bark extract can be assessed using a keratocyte cell culture or reconstructed human epidermis as exemplified herein. Keratocyte cell lines and reconstructed human epidermis are well-known in the art and available from commercial sources. For example, a reconstructed human pigmented epidermis (RHPE, skin type IV), can be obtained from SkinEthic (Lyon, France). This RHPE is characterized as 3-D keratinocyte and melanocyte culture from foreskin disposed on a 0.4 μM TRANSWELL chamber that allows an air-liquid interface.

In accordance with the present invention, a Pinus strobus bark extract exhibiting the desired activities of decreasing endothelin-1 secretion, stem cell factor synthesis and/or protein carbonylation in keratinocytes, is composed of about 4% (and not more than 8%) flavonoids, 15% (and not more than 30%) polyphenols, 0.1% trans-resveratrol, and 0.1% catechin based upon the solid content of the extract. The chemical composition of the Pinus strobes bark extract can be determined as described herein or using any other conventional methodology. For example, total flavonoid content can be assessed using a spectrophotometric method, based on flavonoid-aluminum chloride (AlCl₃) complexation using catechin as a reference compound (Zhishen, et al. (1999) Food Chem. 64:555-559; da Silva, et al. (2015) Pharmacolgn. Mag. 11(41):96-101). Quantification of polyphenols can employ colorimetric methods such as the Folin-Ciocalteu (Singleton et al., (1965) Am. J. Enol. Vitic. 16:144-158) and Prussian-Blue (Budini, et al. (1980) J. Agric. Food Chem. 28(6):1236-8) methods for total polyphenol determinations. The estimation of polyphenols also can be done by, e.g., nuclear magnetic resonance, near-infrared reflectance spectroscopy, high-performance thin layer chromatography (HPTLC), liquid chromatography coupled with mass spectroscopy (LC-MS), high-performance capillary electrophoresis (HPCE) and high-performance liquid chromatography (HPLC), or a combination thereof. The levels of trans-resveratrol can be measured using, e.g., a direct injection isocratic UV-HPLC method (Arslan & Yilmaz (2013) Asian J. Chem. 25(3):1225-8) or electrochemical measurements (Liu, et al. (2017) J. Anal. Methods Chem. 2017:5749025). The amount of catechin present in the extract can be assessed by UV-HPLC (Raju, et al. (2014) Int. Sch. Res. Notices 2014:628196), LC-MS/MS, or a combination thereof (Susanti, et al. (2015) As. Pac. J. Trop. Biomed. 5(12):1046-50).

As indicated herein, the invention provides a method of decreasing endothelin-1 secretion, stem cell factor synthesis and/or protein carbonylation in keratinocytes. The method involves contacting keratinocytes with an aqueous extract of Pinus strobus bark, or optionally a formulation containing the same. Ideally, the extract is topically applied to the skin to have the desired effect on the keratinocytes. In accordance with the method of this invention, an effective amount of the Pinus strobus bark extract is provided to yield a measurable decrease in endothelin-1 secretion, stem cell factor synthesis and/or protein carbonylation by the keratinocytes. In particular embodiments, endothelin-1 secretion is decreased by at least 30%, 40%, 50%, or 60%. In other embodiments, stem cell factor synthesis is decreased by at least 50% or 60%. In further embodiments, protein carbonylation is decreased by at least 10% or 15%.

In addition to the methods disclosed in the examples herein, any conventional method can be used to assess the effects of the Pinus strobus bark extract on keratinocytes. For example, concentrations of endothelin-1 secreted by keratinocytes can be measured using a radioimmunoassay (Ando, et al. (1989) FEBS Lett. 245:164-6) or an enzyme-linked immunoassay (ELISA; Kurita, et al. (2011) Biochem. Biophys. Res. Comm. 409(1):103-7). Stem cell factor synthesis can be measured by western blot analysis or ELISA (Grabbe, et al. (1996) J. Invest. Dermatol. 107:219-224). Protein carbonylation analysis can be carried out by derivatization of the carbonyl group, most commonly with dinitrophenol hydrazine, and quantifying dinitrophenol hydrazone (DNP) adducts by immunoassay (Alamdari, et al. (2005) Free Rad. Biol. Med. 39(10):1362-7; Buss & Winterbourn (2002) Meth. Mol. Biol. 186:123-8). Mass spectrophotometric methods can also be used for identification as well as relative quantification of carbonylated peptides by label-free techniques or using isotopically labelled derivatization reagents.

To facilitate application of the Pinus strobus bark extract to the skin, the present invention also provides preparations or formulations that include the Pinus strobus bark extract. Such compositions may be prepared in various forms, and are desirably prepared in a form that facilitates topical application to the skin. Accordingly, suitable forms of preparation include a cream, ointment, foam, lotion, plaster, gel, solution and emulsion. The frequency of topical applications of such a composition may depend on several factors, including the desired level of suppression of endothelin-1 secretion, stem cell factor synthesis and/or protein carbonylation. The compositions of the present invention can desirably be applied to the skin twice daily, and are particularly desirably applied once in the morning and once in the evening.

The amount of Pinus strobus bark extract present in a formulation will depend upon several factors, including the desired level of activity, the capacity of a particular preparation for a particular extract, and other factors. When used as a fluid (e.g., dissolved in a glycerin solution), the Pinus strobus bark extract is between about 0.01% and about 50% (wt/wt) of the total composition. More desirably, the Pinus strobus bark extract is between about 0.10 and about 25% (wt/wt) of the total composition. Still more desirable, the Pinus strobus bark extract is between about 0.25 and 10% (wt/wt) of the total composition. When used as a solid, the Pinus strobus bark extract is between about 0.001% and 10% (wt/wt) of the total composition. More desirably, the Pinus strobus bark extract is between about 0.002% and 1% (wt/wt) of the total composition. Still more desirable, the Pinus strobus bark extract is between about 0.005 and 0.5% (wt/wt) of the total composition.

A formulation of the present invention is particularly well suited for topical application and for use on human skin. Accordingly, the present invention also includes the cosmetic use of the formulations according to the present invention. Specifically, the present invention includes the cosmetic use of a composition containing an extract of Pinus strobus bark obtained by hot water (i.e., between 70 and 85° C.) extraction of Pinus strobus bark.

A formulation containing a Pinus strobus bark extract may be a viscous or semi-viscous fluid, or a less viscous fluid such as might be used in sprays or aerosols. It may take the form of a solution, suspension or emulsion. It may take the form of a solid such as a powder or granules, which may be designed to be added to liquid (e.g., water) prior to use. In some embodiments the formulation is, or may be, applied to a carrier such as a sponge, swab, brush, pad, tissue, cloth, wipe, skin patch or dressing (which includes a bandage, plaster, skin adhesive or other material designed for application to a tissue surface), to facilitate its administration.

A formulation according to the invention may contain excipients and other additives known for use in topical formulations. Suitable excipients for use in formulations designed for topical or local application will be well known to those skilled in the art. Those included will depend on the intended mode and site of application for the formulation. In the context of formulations for topical application to the skin, examples may for instance be found in Williams' Transdermal and Topical Drug Delivery (Pharmaceutical Press, 2003) and other similar reference books. See also Date, et al. ((2006) Skin Pharmacol. Physiol. 19(1):2-16) for a review of topical delivery strategies, and also Skin Delivery Systems ((2006) John J Wille, Ed, Blackwell Publishing.

Where the formulation is intended for topical application to the skin, examples of suitable additives include emollients, moisturizers, perfumes, antioxidants, preservatives, stabilizers, gelling agents and surfactants; others may be found in Williams' Transdermal and Topical Drug Delivery (see above). Any additional additives used in the compositions must not be irritating, and should not detrimentally affect the desired activities of decreasing endothelin-1 secretion, stem cell factor synthesis and/or protein carbonylation in keratinocytes.

The Pinus strobus bark extract may be used individually, or may be used in combination with one or more additional active ingredients such as antimicrobial agents, anti-inflammatory agents, anti-acne agents, keratolytics, comedolytics, agents capable of normalizing keratinocyte and/or sebocyte function, anti-inflammatories, anti-proliferatives, anti-androgens, sebostatic/sebosuppressive agents, anti-pruritics, immunomodulators, anti-irritant, agents which promote wound healing, sunscreens, skin lightening agents, anti-aging substances, and mixtures thereof.

A formulation of use in the methods of this invention may be incorporated into, and hence applied in the form of, a cosmetic; a skin care preparation (for example a skin cleanser, toner or moisturizer); a cleansing preparation (for example a facial wash or scrub); a cosmeceutical preparation; a toiletry product (for instance a bath or shower additive or a soap). The formulation may be, or be incorporated into, a wash-off skin treatment product such as a skin cleanser, or in particular a leave-on skin product.

The invention is described in greater detail by the following non-limiting examples.

### Example 1: Extraction and Chemical Composition of Pinus strobus Bark Extract

Extraction Process. The extract was prepared using exclusively residual Pinus strobus barks from the Canadian forestry industry. The barks were collected then dried to a moisture content of less than 10%. Once dry, they were milled in a first hammer mill to 2 mm, then milled again and screened to 0.5 mm. The homogeneous bark material was then extracted by maceration in hot water following a specified set of parameters. More specifically, the plant material was first mixed with the water at a ratio of 1:16 by weight. The mixture was brought to 85° C., heat was removed and the mixture was left at temperature for a full hour under agitation. After the extraction was complete, a first separation of the solids and liquids was conducted via a continuous centrifuge separator followed by removal of the fines using a press filter. The resulting clear extract was concentrated under vacuum and low heat, and subsequently flash pasteurized. The concentrated extract was put under vacuum and low heat for many hours for complete evaporation of the water, which resulted in a dry powder. The powder, which was deemed a pure extract, was subsequently dissolved in a mixture of water and glycerin under strong agitation and moderate heat for a few hours. That mixture was used in subsequent analyses and final product preparation.

Chemical Composition. The Pinus strobus extract was monitored for its chemical properties during and after its preparation in order to assess its quality and usability. The chemical properties included the amounts of polyphenols, flavonoids, catechin and trans-resveratrol. These properties were used to ensure reproducibility between batches and to provide an overall quantifiable quality of the product. As is conventional in the art, the concentration of total flavonoid content was calculated from a calibration plot (5-200 μg/mL quercetin) and expressed as a quercetin equivalent. Likewise, the phenolic content was calculated as gallic acid equivalents on the basis of a standard curve of gallic acid (5-500 mg/L). See, e.g., Chandra, et al. (2014) Evidence-Based Complementary and Alternative Medicine, vol. 2014, Article ID 253875). Chemical composition analysis of the Pinus strobus extract indicated that extracted contained 4% flavonoids (quercetin equivalents), 15% polyphenols (gallic acid equivalents), 0.1% trans-resveratrol, and 0.1% catechin.

### Example 2: Effect of Pinus strobus Bark Extract on Melanin Synthesis

Skin pigmentation is the result of melanin synthesis by melanocytes through melanogenesis. Once synthesized, melanin is transferred trough keratinocytes into vesicle called melanosomes. Epidermal turnover then brings the melanin pigment up to the surface and the skin pigmentation appears. In order to investigate physiological activity of Pinus strobus bark extract on skin pigmentation, the capacity of the extract to reduce melanin production was assessed using B16-F1 cell cultures.

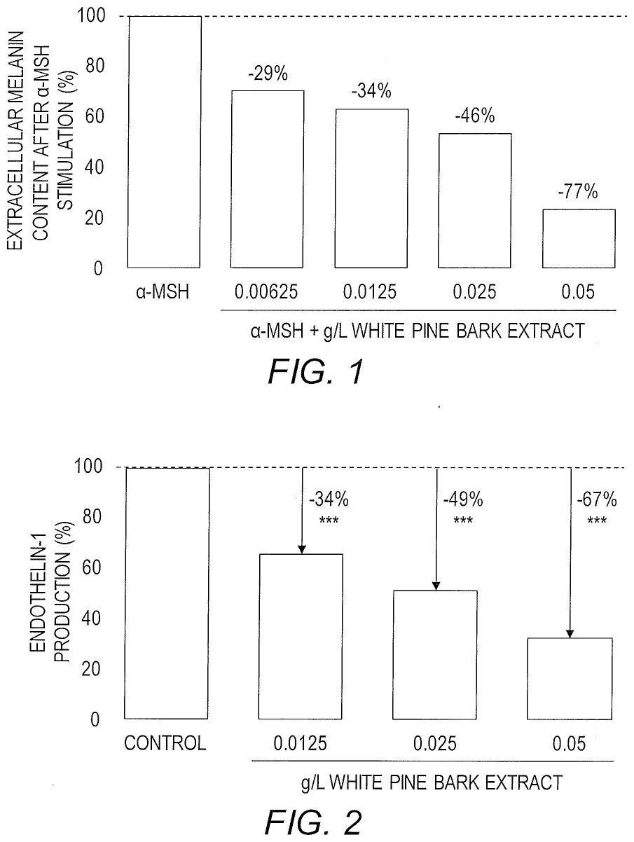

B16-F1 cells (a murine melanocytes cell line) were maintained in Dulbecco's Modified Eagle's Medium (DMEM) containing 10% fetal calf serum, 1% antibiotics (penicillin/streptomycin) and 1% L-glutamine at 37° C. under 5% CO₂ and 95% humidity. B16-F1 cells were seeded in 96-well plates at 1.5×10⁴ cells/well. After 24 hours, 20 ng/ml α-MSH was added to induce the melanogenesis. At the same time, Pinus strobus bark extract (0.00625 g/L, 0.0125 g/L, 0.025 g/L and 0.05 g/L) was added and melanogenesis synthesis was measured based upon the release of melanin in the extracellular medium. Melanin concentration was measured using a spectrophotometer at 405 nm and the results are indicated as percent activation of melanin synthesis by α-MSH. All experimental conditions were performed in n=3. This analysis indicated that after 4 days of α-MSH stimulation, Pinus strobus bark extract provided a dose-dependent decrease in melanin synthesis (FIG. 1).

### Example 3: Effect of Pinus strobus Bark Extract on Endothelin-1 Synthesis

Melanogenesis is induced by paracrine factors secreted by endothelial cells. Endothelin-1, a protein synthesized by keratinocytes, binds to the specific endothelin B (ET_{B}) receptor on melanocyte membranes and induces melanin production (Imokawa & Ishida (2014) Int. J. Mol. Sci. 15:8293-8315). Accordingly, the effect of Pinus strobus bark extract on endothelin-1 synthesis by human keratinocytes was assessed.

Normal Human Epidermal Keratinocytes (NHEK) were maintained and cultured in EPILIFE® medium supplemented with CaCl₂ (Life Technologies), at 37° C., 5% CO₂, and 95% humidity. NHEK cells were seeded in 96-well plates at a concentration of 2×10⁴ cells/ml for 24 hours. After 1 day, the medium was removed and cells were treated with different concentrations of Pinus strobus bark extract (0.0125 g/L, 0.025 g/L and 0.05 g/L) for an additional 24 hours. Subsequently, supernatants were collected and Endothelin-1 release from keratinocytes was determined by using an Endothelin Pan Specific ELISA (enzyme-linked immunosorbent assay; R&D Systems, Minneapolis, Minn.) following the manufacturer's protocol.

The results are presented as the percentage of Endothelin-1 secretion (FIG. 2) and indicate that Pinus strobus bark extract exhibits a dose-dependent inhibition of Endothelin-1 synthesis.

### Example 4: Effect of Pinus strobus Bark Extract on Stem Cell Factor Synthesis

Similar to Endothelin, stem cell factor (SCF) is induced by paracrine factors and secreted by keratinocytes. SCF binds to the c-Kit receptor on melanocyte membranes and induces melanin production (dos Santos Videira, et al. (2013) An. Bras. Dermatologie 88(1):76-83). Accordingly, the effect of Pinus strobus bark extract on SCF synthesis by human keratinocytes was assessed.

NHEK cells were maintained and cultured in EPILIFE® medium supplemented with CaCl₂ (Life Technologies), at 37° C., 5% CO₂, and 95% humidity. NHEK cells were seeded in 96-well plates at a concentration of 2×10⁴ cells/ml for 24 hours. After 1 day, the medium was changed and the cells were allowed to grow for 6 hours. Subsequently, the cells were treated with different concentrations of Pinus strobus bark extract (0.025 g/L and 0.05 g/L) for an additional 24 hours. The supernatants were collected and SCF release was determined using a Human SCF ELISA Development kit (PromoKine) following the manufacturer's protocol.

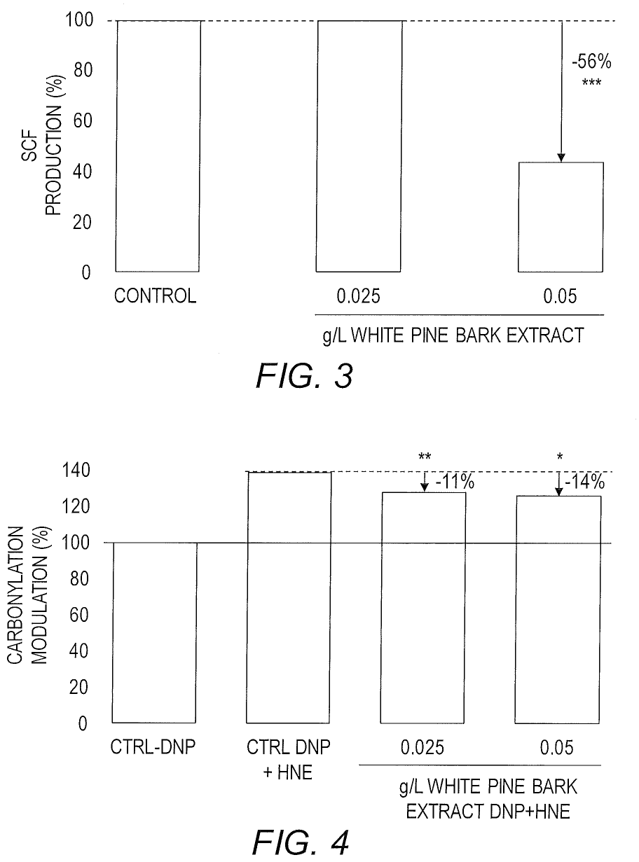

This analysis indicated that at 0.05 g/L the Pinus strobus bark extract inhibited SCF synthesis by 56% (FIG. 3).

### Example 4: Effect of Pinus strobus Bark Extract on Protein Carbonylation

Protein carbonylation is a biomarker of oxidative stress in dermal cells. Detected in the stratum corneum (SC) exposed to external stress, protein carbonylation induces change in the water holding capacity but also in the optical property of SC which influences skin transparency (Iwai, et al. (2008) Int. J. Cosmet. Sci. 30(1):41-46). Furthermore, analysis of photo-aged dermal skin shows a correlation between yellowish color and carbonyl modification (Ogura, et al. (2011) J. Dermatol. Sci. 64(1):45-52). Notably, carbonylation can be detected and quantified at the global level in proteins and protein mixtures using derivatization of carbonyl groups with 2,4-dinitrophenylhydrazine (DNPH) followed by spectrophotometric measurements or immunodetection with DNPH-specific antibodies either in gels or in ELISA assay (Rogowka-Wrzesinska, et al. (2014) Free Rad. Res. 48(10):1145-62). According, using DNPH, the effect of Pinus strobus bark extract on protein carbonylation in human keratinocytes was assessed.

NHEK cells were maintained and cultured in EPILIFE® medium supplemented with CaCl₂ (Life Technologies), at 37° C., 5% CO₂, and 95% humidity. NHEK cells have been seeded in 6-well plates at a concentration of 3×10⁵ cells/ml. At 70-80% confluency, cells are treated with Pinus strobus bark extract at the different concentrations (0.025 g/L and 0.05 g/L). After 24 hours, medium was removed and the cells were treated with 20 μM 4-Hydroxynonenal (HNE; a known activator of protein carbonylation) for an additional 24 hours. At the end of the incubation, the cells were rinsed, lysed for protein extraction and quantification (Pierce, Thermo Fisher scientific). Protein carbonylation was determined using an Oxyblot™ Protein Oxidation Detection kit (Millipore) including DNPH (1 μL DNPH per 0.5 μg/μL of proteins). DNP-derivatized proteins were detected using an anti-DNP primary antibody and an HRP-conjugated secondary antibody. Antibody binding was quantified on Image J software based upon chemiluminescence intensity normalized to protein concentration. All experimental conditions were performed in n=3.

This analysis indicated that at the Pinus strobus bark extract significantly inhibited protein carbonylation by at least 11% (FIG. 4). In light of these results, the Pinus strobus bark extract is of use in improving skin complexion.

### Example 5: Evaluation of Melanin Synthesis in an Ex Vivo Skin Model

Paracrine factors secreted by keratinocytes have been shown to activate specific melanogenesis receptors on melanocytes, thereby modulating melanin production. To demonstrate that the Pinus strobus bark extract inhibits melanin synthesis, the effect of the extract in a formulation was analyzed on pigmented reconstructed epidermis.

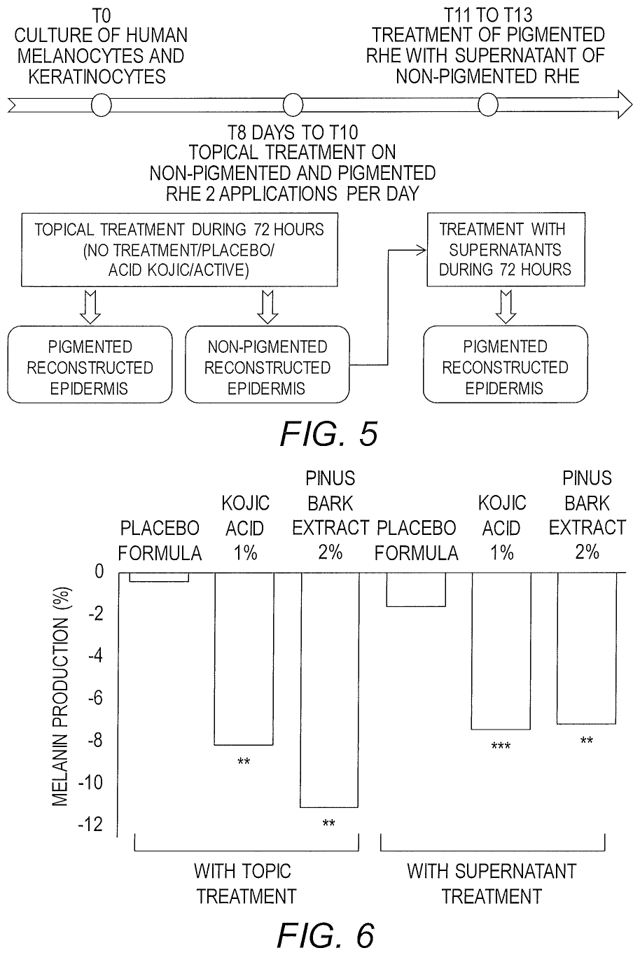

Non-pigmented reconstructed human epidermis (RHE) and pigmented RHE phototype IV were prepared by seeding human melanocytes and keratinocytes in an incubation chamber placed on the epidermal side of a dead dermis at 4×10⁵ cells per cm², at a 1:20 melanocyte:keratinocyte ratio. After 24 hours, the incubation chamber was removed and the dead dermis was submerged for 3 days. The dead dermis was moved to the air-liquid interface for 8 days prior to treatment. After 8 days of exposure to the air-liquid interface, the pigmented RHE and non-pigmented RHE were contacted with Pinus strobus bark extract, placebo, negative control (no treatment), or positive control (kojic acid). This involved topical application of each formulation for 3 days, 2 applications per day (see FIG. 5). After 3 days, pigmented RHE was incubated for 3 days in the presence of a 1:1 mixture of fresh media and conditioned media from the non-pigmented RHE.

The formulations included Heliogel™ (sodium acrylates copolymer, hydrogenated polyisobutene, phospholipids, polyglyceryl-10 stearate, sunflower seed oil; Lucas Meyer Cosmetics), Saboderm TCC (Caprylic/Capric Triglyceride; SABO, S.p.A), and Dekaben C (phenoxyethanol, methylparaben, ethylparaben, butylparaben, isobutylparaben, propylparaben; Jan Dekker BV) in the amounts listed in Table 1.

TABLE 1

| | Formulation (wt/wt) | | |
|---|---|---|---|
| | | Kojic | Pinus strobus |
| Ingredient | Placebo | acid | bark extract |
| Heliogel ™ | 3% | 3% | 3% |
| Saboderm TCC | 15% | 15% | 15% |
| Water | 81.2% | 80.2% | 79.2% |
| Dekaben C | 0.8% | 0.8% | 0.8% |
| Kojic acid | — | 1% | — |
| Pinus strobus bark | — | — | 2% |
| extract | | | |

Pigmentation was evaluated by Fontana-Masson staining. RHE sections were also examined by light microscopy. As a highly concentrated melanin region creates high intensity dark signals, an increase in luminosity exhibits a decrease in melanin content.

This analysis indicated that topical treatment of the pigmented RHE with Pinus strobus bark extract resulted in a significant decrease in melanin synthesis (11.2%), which exceeded that provided by Kojic acid (8.1%) at twice the dose (FIG. 6). Pigmented RHE treated with the supernatant of non-pigmented RHE also resulted in a significant decrease in melanin synthesis (7.2%) when the non-pigmented RHE was treated with Pinus strobus bark extract. This indicates that Pinus strobus bark extract acts directly on keratinocytes and modulates secretion of regulatory molecules required for melanogenesis.

What is claimed is:

1. A method for decreasing endothelin-1 secretion, stem cell factor synthesis or protein carbonylation in a keratinocyte comprising contacting a keratinocyte with an effective amount of an aqueous extract of Pinus strobus bark thereby decreasing endothelin-1 secretion, stem cell factor synthesis or protein carbonylation in the keratinocyte.

2. The method of claim 1, wherein endothelin-1 secretion is decreased by at least 30%.

3. The method of claim 1, wherein stem cell factor synthesis is decreased by at least 50%.

4. The method of claim 1, wherein protein carbonylation is decreased by at least 10%.

5. The method of claim 1, wherein the aqueous extract of Pinus strobus bark comprises about 4% flavonoids, 15% polyphenols, 0.1% trans-resveratrol, and 0.1% catechin.

6. The method of claim 1, wherein the aqueous extract of Pinus strobus bark is in the form of a cream, ointment, foam, lotion, plaster, gel, solution or emulsion.
